(19) United States
(12) Patent Application Publication
(10) Pub. No.: US 2002/0010152 A1
(43) Pub. Date: Jan. 24, 2002

(54) COMPOSITION CONSISTING OF HEPARIN FRACTIONS HAVING REPRODUCIBLE CHARACTERISTICS WITH AVERAGE MOLECULAR WEIGHT EQUAL TO 5200D

(76) Inventors: LUIGI DE AMBROSI, SANTHIA (IT); SERGIO GONELLA, SANTHIA (IT); FRANCESCA MARCENARO, NOVI LIGURE (IT)

Correspondence Address: JAMES V COSTIGAN, HEDMAN GIBSON AND COSTIGAN, 1185 AVENUE OF THE AMERICAS, NEW YORK, NY, 10036

(21) Appl. No.: 09/213,538

(22) Filed: Dec. 17, 1998

(30) Foreign Application Priority Data
Dec. 19, 1997 (IT) MI97A002835

(51) Int. Cl. C08B 37/08; A61K 31/727

(52) U.S. Cl. 514/56; 536/20

(57) ABSTRACT

Composition consisting of heparin fractions having reproducible characteristics with average molecular weight of 5,200 D obtained by depolymerization with gamma radiation and subsequent fractionation by gel permeation, having high antithrombotic properties and particularly suitable for the prophilaxis and the therapy of the alterations of the plasmatic homeostasis.

### PRIOR ART

Processes to obtain the heparin and glycosaminoglycans derivatives having in general low molecular weight, for example from 5,000 to 8,000 D, are known.

A process is described for example in U.S. Pat. No. 4,987,222 wherein gamma radiations from Cobalt-60 are used for the depolymerization of the heparin and other glycosaminoglycans for the achievement of derivatives having low molecular weight.

Other processes are based on chemical or enzymatic treatments obtained using nitrous acid, with a benzilation intervention followed by alkaline hydrolysis, digestion with isoamyl nitrite, peroxidative breaking, the use of the specific heparinases.

The process based on the treatment with gamma radiations shows the advantage to maintain unaltered, in the obtained fractions, the originary structures while in the processes based on chemical or enzymatic reactions such structures are altered whereby further treatments of semisynthesis and resulfation turn out to be necessary.

In any case, the problem of individuating and separating fractions having specific activities able to intervene with aimed effects in the different antithrombotic therapies is still open. Moreover the therapeutic use of a mixture of fractions having low molecular weight in order to carry on a constant activity needs a continuative and repeatable composition of those fractions able to express the maximum pharmacological intervention in the absence of toxic phenomenologies typical of particular molecular weights.

### SUMMARY

Now we have found a composition consisting of heparin fractions having high antithrombotic properties and particularly suitable for the prophylaxis and the therapy of the alterations of the plasmatic homeostasis.

Said composition is characterized by an average molecular weight equal to 5,200 D (±500) with a polydispersion index ranging from 1.04 to 1.10, and characterized in that it consists of the following fractions:

| | | |
|---|---|---|
| 7,000 D (±500) | 11-15% | by weight |
| 6,000 D (±500) | 14-16% | by weight |
| 5,000 D (±500) | 28-35% | by weight |
| 4,000 D (±500) | 23-27% | by weight |
| 3,000 D (±500) | 13-15% | by weight |

Said fractions maintain unaltered the original structures of the heparin, in particular the value of the sulfation indexes constituting the indispensable elements for an effective therapeutic action is maintained unaltered.

The composition according to the present invention is obtained with constant and reproducible characteristics from the qualitative and quantitative point of view starting from sodium heparin of animal extraction having an average molecular weight equal to 13,000-15,000 D by treatment with gamma radiations and subsequent fractionation by gel permeation.

### BRIEF DESCRIPTION OF THE DRAWINGS

FIG. 1 represents the NMR characteristics of the composition according to the present invention (GAMMAPARIN).

FIG. 2 represents the NMR characteristics of the depolymerized mother heparin.

FIG. 3 represents the NMR characteristics of the starting heparin.

FIG. 4 represents the HPLC determination of the composition according to the present invention (GAMMAPARIN).

FIG. 5 represents the HPLC determination of the depolymerized mother heparin.

FIG. 6 represents the HPLC determination of the starting heparin.

### DETAILED DESCRIPTION OF THE INVENTION

The characteristics and the advantages of the composition consisting of heparin fractions obtained from depolymerization by treatment with gamma radiation according to the present invention, will be mostly shown during the following detailed description.

The starting material for the preparation of said composition consists of sodium heparin obtained from animal extraction having an average molecular weight equal to 13,000-15,000 D, activity equal to 180-190 Ul/mg and without EDTA, heavy metals and solvents.

An aqueous solution of said heparin having a concentration ranging from 5 to 15% weight/volume is prepared.

The solution, in a neutral glass container, is piped to the irradiation cell by a system controlled by a software.

The irradiation system includes a 5 mCi radioactive source of Cobalt-60 and the heparin solution is submitted to a treatment ranging from 120 kGy to 150 kGy with subsequent dosages of about 25 kGy, depending on the molecular weight of the starting heparin.

The irradiated solution is treated with cut ultrafiltration 300 D and then it is purified, concentrated, filtered and freeze-dried.

The freeze-dried product is indicated as “depolymerized mother heparin”.

The freeze-dried product is dissolved in an aqueous solution of 0.3 M NaCl in such an amount to obtain a concentration ranging from 10 to 15% weight/volume and the obtained solution is treated with a gel permeation process for the fractionation.

Said process is carried out in a column containing G/50 medium kind Sephadex resin having a granulometry ranging from 50 to 150 micrometers, able to separate fractions having molecular weight ranging from 1,000 to 10,000 D.

For example a BP 252/15 kind pilot column may be used having the following characteristics:

height: 105 cm;

resin volume: 52 l;

flux: 2 l/h of 0.3 M NaCl.

Working with the following parameters:

Ve=17 l;

Ve/Vo=1/R=1.26;

R=0.79;

K=(Ve-Vo)/(Vt-Vo)=0.09

wherein the elution volume is indicated with Ve,

the dead volume is indicated with Vo,

the retention constant is indicated with R,

the total volume of the resin bed is indicated with Vt,

17 fractions having highly reproducible characteristics and molecular weights ranging from values lower than 3,000 D to values higher than 9,000 D are usually produced.

The present invention relates to the mixture of fractions having the following composition:

| Molecular Weight | % by weight |
|---|---|
| 7,000 D (±500) | 11-15 |
| 6,000 D (±500) | 14-16 |
| 5,000 D (±500) | 28-35 |
| 4,000 D (±500) | 23-27 |
| 3,000 D (±500) | 13-15 |

This mixture represents about the 50% by weight of the total of the produced fractions.

Said mixture, which below will be indicated as GAMMAPARIN, shows biological and therapeutic unexpected activities with the absence of haemorrhagic and allergic effects.

The chemical characteristics of GAMMAPARIN are reported in Tables 1 and 2.

TABLE 1

| | |
|---|---|
| Average Molecular Weight | 5,200 ± 500 |
| Polydispersion Index | 1.04-1.10 |
| Absorbance at 260 nm | <0.200 |
| Absorbance at 280 nm | <0.150 |
| 1% Solution pH | 5.5-8.0 |
| N % | 1.5%-2.5% |
| Dying Loss | <5% |
| Organic S | >10% |
| Na % | 9.5%-12.5% |
| SO₃/COOH | >2 |

TABLE 2
(with reference to the starting heparin)
Ratios of the content in sulfated organic groups obtained by NMR analysis

| | Initial Heparin | Gammaparin |
|---|---|---|
| (1) Desulfated Uronic | 35-38% | 31-34% |
| Acids | | |
| (2) GlcNSO₃6SO₃ | 81-85% | 83-86% |
| (3) GlcNAc | 12-15% | 12-16% |
| (4) GlcNSO₃3SO₃ | 6-7% | 7-8% |

wherein (1) is the ratio between the desulfated uronic acids with respect to the total uronic acids, (2) is the ratio between sulfated 6 glucosamine and the not sulfated one, (3) is the ratio between the N acetylated glucosamines and the sulfated ones, (4) is the ratio between the N,3 sulfated glucosamine and the not sulfated one.

These ratios confirm the validity of the depolymerization obtained without chemical interventions of molecular adjustment.

The process based on the treatment with gamma radiations according to the present invention in fact has the advantage to maintain, in the obtained fractions, the original structures unaltered while in the processes based on chemical or enzymatic reactions such structures are often altered whereby further semisynthesis and resulfation treatments are needed.

The majority of the GAMMAPARIN components shows in the not reducing terminal of the chain a 2-O-sulfo-a-L-idopyranosuronic acid and in the reducing terminal of the chain a 2-N, 6-O-disulfo-D-glucosamine, as it is shown in the structure reported below:

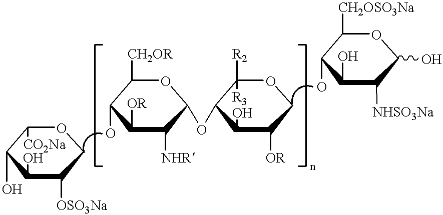

with n ranging from 1 to 21, R=H or SO₃Na, R′=SO₃Na or COCH₃, R₂=H and R₃=CO₂Na or R₂=CO₂Na and R₃=H.

The process according to the present invention carries on its intervention exclusively on the desulfated units of the structural components of the heparin molecule and it points out the possibility to obtain a high reproducibility in the formation of the derivatives having lower molecular weights in total absence of impurities formed by catalysts and by organic and inorganic chemical compounds extraneous to the heparin composition.

The biological activity profiles are reported in Table No. 3

TABLE No. 3

| | APTT | antiXa | antilla |
|---|---|---|---|
| | U/mg | UaXa/mg | Ualla/mg |
| GAMMAPARIN | 25-60 | 80-100 | 30-50 |

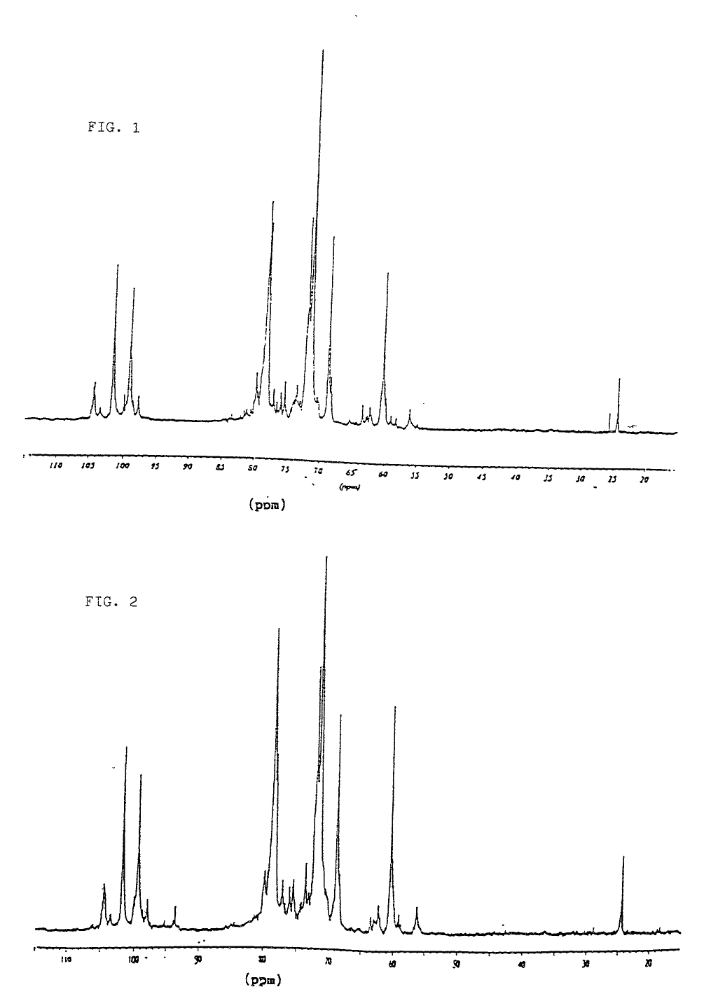

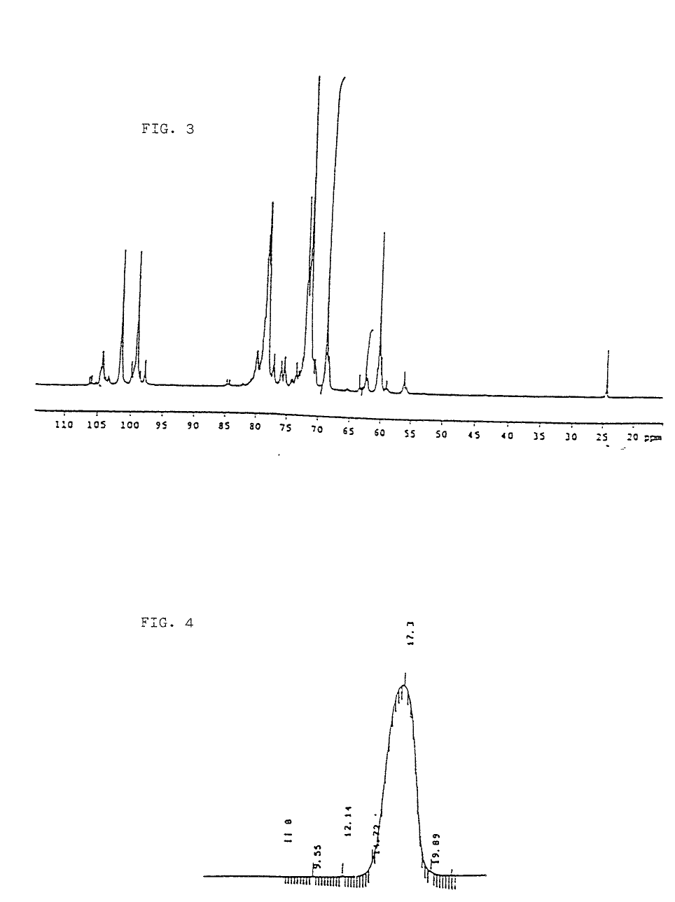

The structural characteristics of the GAMMAPARIN, determined by NMR are reported in the FIG. 1 in comparison with the FIG. 2 of the Depolymerized Mother Heparin and in comparison with the FIG. 3 of the starting Heparin. In the FIG. 1 (GAMMAPARIN) the signals between 84 and 85 ppm characteristic of the binding site which is removed by the gamma depolymerization do not occur. The detachment of the galactosidic chain and its nitrogenous components represents a specific characteristic of this fraction able to work without interferences, often of pathological character, deriving from the presence of peptidic structures having different aminoacidic composition.

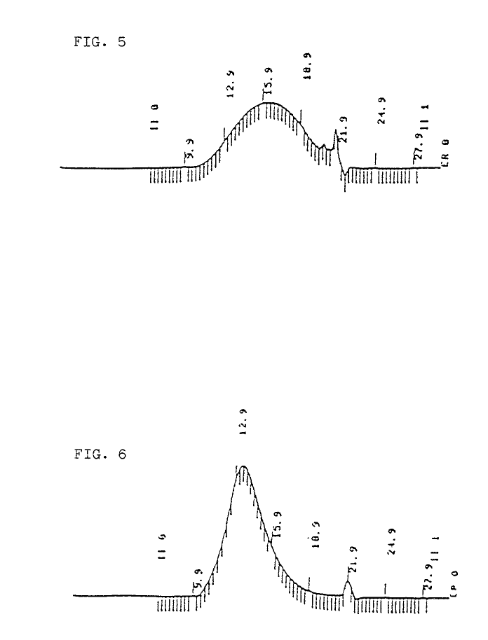

The plots of the determinations in HPLC corresponding to the FIG. 4 for the GAMMAPARIN, to the FIG. 5 for the Depolymerized Mother Heparin and to the FIG. 6 for the starting heparin are reported too.

### PHARMACODYNAMICAL EXPERIMENTATIONS

The GAMMAPARIN antithrombotic activity has been tested according to “Rabbit Jugular-Vein Thrombosis Model” according to Friedman and coll. (Am. J. Physiol. 199: 750 1960).

The intravenous administration (IV) of GAMMAPARIN from this test turned out to show a ED/50 equal to 2.15 mg/kg based on administrations equal to 1 and 5 mg/kg showing an evident antithrombotic potential as compared with the ED/50 of the initial heparin equal to 0.3 mg/kg. The “Bleeding Time” test has been carried out by the “Tail Transection” method according to Dejana and col. (Thromb. Haemostasis 48: 108: 1982). The bleeding times of the GAMMAPARIN in comparison with the starting heparin at the dose of 0.5 mg/kg administered 15′ before the cut are indicated in the following ranges:

Physiological Solution=155″±12.5

GAMMAPARIN=288″±15

Starting heparin=715″±43

These values point out the effect of the particular composition of the GAMMAPARIN which shows a low bleeding index with respect to the high values of the heparin for the total absence of fractions having a molecular weight higher than 9,000 D.

These results confirm the structural validity of the GAMMAPARIN components, identifiable with the primary natural elements recognized as the only active factors for the biological functions of the organism obtained from biological depolymerization of a highly polymerized component.

These results bear out the validity of the physical process of the decomposition of the heparin polymeric structure according to the present invention which is based on schemes of not destructive intervention assimilable to the biological phenomena of natural molecular scission.

All the technical-biological data reported in the present Patent are obtained from GAMMAPARIN having sodium salt form, but they may be obtained also from the corresponding calcium, potassium, lithium, magnesium salts.

Moreover a mixture of analogous fractions may be obtained starting from other glycosaminoglycans such as Dermatan, Chondroitin, Heparan, Heparide and generally heparinoids.

The GAMMAPARIN may be used, as a single substance or in mixture with other therapeutic substances, for the preparation of pharmaceutical compositions for subcutaneous, intramuscular, intravenous, topical and aerosol administration, in mixture with pharmaceutically acceptable solvents or excipients.

Said pharmaceutical compositions turn out to be suitable to the prevention of the postoperative thromboembolisms, to the prophylaxis of the uremic patients submitted to chronical dialysis, to the prevention and the therapy of the deep venous thromboses and generally toward the alterations of the plasmatic homeostasis.

In particular, as far as the deep venous thrombosis prevention is concerned the administration by subcutaneous injection of 100 Ul per kg of body weight every 12 hours for at least 10 days is contemplated.

For the prevention of the postoperative thromboembolisms the subcutaneous administration of 2,500-5,000 Ul every 24 hours for at least 7 days is contemplated.

For illustrative aim the following Examples of preparations which, according to the present invention, maintain constant the quali-quantitative composition of the fractions by a technology carrying out a radiation intervention as a function of the molecular weight of the starting heparin are reported.

### EXAMPLE 1

100 g of sodium heparin obtained from animal extraction and having an activity equal to 190 Ul/mg and M. W.=15,175 D (composition percent of the fractions in Table A) are dissolved in 1 l of bidistilled and de-aerated water. The solution is poured into a Pyrex container which is closed by a glass plug after bubbling with argon.

The solution is treated with a total dose of 130 kGy at subsequent dosages of about 25 kGy of gamma radiation from Co60.

The irradiated solution is treated with 300 D cut ultrafiltration and purified with subsequent passages in 3% NaCl. The solution concentrated at 10% is freeze-dried and the freeze-dried product (Depolymerized Mother Heparin No. 1) is dissolved at 10% in 0.3 M NaCl solution and fractionated with gel permeation column on G/50 medium Sephadex (batch No. 1/A).

The Table B reports the values percent of the obtained fractions.

TABLE A
Batch n.1 - MW 15,175 D

| MW | |
|---|---|
| (KD ± 500) | % Fractions |
| >20 | 23.68 |
| 15 ÷ 20 | 23.18 |
| 15 ÷ 12 | 14.53 |
| 12 ÷ 10 | 11.74 |
| 10 ÷ 8 | 12.41 |
| 8 ÷ 6 | 7.42 |
| 6 ÷ 4 | 5.53 |
| 4 ÷ 2.5 | 1.47 |
| <2.5 | 0.04 |

TABLE B
Batch n.1/A - MW 6,460 D

| MW (D ± 500) | % Fractions |
|---|---|
| >9,000 | 23.21 |
| 8,000 | 7.55 |
| 7,000 | 8.41 |
| 6,000 | 9.05 |
| 5,000 | 18.90 |
| 1,000 | 17.10 |
| 3,000 | 9.31 |
| <3,000 | 6.40 |

The preparation of the composition according to the present invention (GAMMAPARIN) is obtained by the mixing of the fractions ranging from 7,000 D to 3,000 D in order to obtain an average molecular weight equal to 5,200 D (±500). The mixture in an aqueous solution at a concentration equal to 5 g/l is submitted to desalting on 1,000 D cut-off membranes and then it is concentrated to a concentration equal to 50 g/l. The concentrated solution is freeze-dried after undergoing sterile filtration.

About 40 g of a composition with the following characteristics are obtained:

Average M. W. 5,200 D (±500)

Molecular Weights Distribution: <3,000 D=1.5% ->7,000 D=8%

Polydispersion Index: <1.1

Sulfates/Carboxyl Ratio: >2

AntiXa Activity: >80 U antiXa/mg.

### EXAMPLE 2

100 g of sodium heparin obtained from animal extraction and having an activity equal to 186 Ul/mg and M. W.=14,200 Da (composition in Table C) are dissolved in 1 l of bidistilled and de-aerated water. The solution is poured into a Pyrex container and bubbled with argon. The closure of the container by a glass plug is immediate. The solution is treated with a total dose of 125 kGy at subsequent dosages of gamma radiation from Cobalt 60. The operations described in the Example No. 1 follow obtaining the Depolymerized Mother Heparin which is fractionated (batch No. 2/A) with the percentages of the fractions of Table D.

TABLE C
Batch n.2 - MW 14,200 D

| MW | |
|---|---|
| (KD ± 500) | % Fractions |
| >20 | 20.18 |
| 15 ÷ 20 | 18.80 |
| 15 ÷ 12 | 20.05 |
| 12 ÷ 10 | 11.70 |
| 10 ÷ 8 | 9.64 |
| 8 ÷ 6 | 10.69 |
| 6 ÷ 4 | 6.76 |
| 4 ÷ 2.5 | 2.08 |
| <2.5 | 0.20 |

TABLE D
Batch n.2/A - MW 6,944 D

| MW (D ± 500) | % Fractions |
|---|---|
| >9,000 | 27.20 |
| 8,000 | 7.77 |
| 7,000 | 8.45 |
| 6,000 | 9.08 |
| 5,000 | 16.10 |
| 4,000 | 15.75 |
| 3,000 | 8.24 |
| <3,000 | 5.56 |

The preparation of the composition according to the present invention (GAMMAPARIN) is obtained by the mixing of the fractions ranging from 7,000 D to 3,000 D in order to obtain an average molecular weight equal to 5,200 D (±500). The operations described in the Example No. 1 follow and a product having the same characteristics is obtained.

### EXAMPLE 3

100 g of sodium heparin obtained from animal extraction and having an activity equal to 185 Ul/mg and M.W.=12,910 D (composition in Table E) after the dissolution operations, are irradiated with a total dose equal to 120 kGy according to the conditions of the Example 1. The Depolymerized Mother Heparin, after fractionation with gel permeation (batch No. 3/A) shows the fractions indicated in Table F.

TABLE E
Batch n.3 - MW 12,910 D

| MW | |
|---|---|
| (KD ± 500) | % Fractions |
| >20 | 16.36 |
| 15 ÷ 20 | 21.23 |
| 15 ÷ 12 | 14.98 |
| 12 ÷ 10 | 12.67 |
| 10 ÷ 8 | 9.86 |
| 8 ÷ 6 | 10.10 |
| 6 ÷ 4 | 7.17 |
| 4 ÷ 2.5 | 3.71 |
| <2.5 | 3.72 |

TABLE F
Batch n.3/A MW 6,800 D

| MW (D ± 500) | % Fractions |
|---|---|
| >9,000 | 26.98 |
| 8,000 | 6.89 |
| 7,000 | 7.27 |
| 6,000 | 9.08 |
| 5,000 | 19.64 |
| 4,000 | 12.96 |
| 3,000 | 8.30 |
| <3,000 | 7.88 |

The preparation of the composition according to the present invention (GAMMAPARIN) is obtained by the mixing of the fractions ranging from 7,000 D to 3,000 D in order to obtain an average molecular weight equal to 5,200 D (±500). The operations described in the Example No. 1 follow and a product having the same characteristics is obtained.

1. Composition consisting of heparin fractions having reproducible characteristics obtained by depolymerization with gamma radiation and subsequent fractionation by gel permeation, having an average molecular weight equal to 5,200 D (±500) and consisting of 11-15% of the fraction having molecular weight equal to 7,000 D (±500), of 14-16% of the fraction having molecular weight equal to 6,000 D (±500), of 28-35% of the fraction having molecular weight equal to 5,000 D (±500), of 23-27% of the fraction having molecular weight equal to 4,000 D (±500) and of 13-15% of the fraction having molecular weight equal to 3,000 D (±500).

2. Composition as claimed in claim 1, having a polydispersion index of the molecular weights ranging from 1.04 to 1.1.

3. Composition as claimed in claim 1, having an absorbance at 260 nm<0.200 and an absorbance at 280 nm<0.150.

4. Composition as claimed in claim 1, wherein it maintains the sulfation values corresponding to the values of the starting heparin.

5. Composition as claimed in claim 1, having an organic sulphur content >10%.

6. Composition as claimed in claim 1, wherein the galactosidic chain and its nitrogenous components present in the starting heparin structure are absent.

7. Composition as claimed in claim 1, having an activity in APTT, antiXa and antilla respectively of 25-60 U/mg, 80-100 UaXa/mg and 30-50 Ualla/mg.

8. Composition as claimed in claim 1, having an antithrombotic activity, ED/50 of 2.15 mg/kg.

9. Composition as claimed in claim 1, having a bleeding index of 288±15 sec. at the dose of 0.5 mg/kg.

10. Composition as claimed in claim 1, being salified with a cation selected from the group consisting of sodium, potassium, lithium, calcium and magnesium.

11. Composition as claimed in claim 1, wherein said fractions derive from Dermatan, Chondroitin, Heparan, Heparide and heparinoids.

12. Pharmaceutical compositions suitable to the prophylaxis and the therapy of the plasmatic homeostasis alterations containing an effective amount of the composition as claimed in claim 1, either as a single substance or in association with other therapeutic substances.

13. Pharmaceutical compositions including an effective dose of the composition as claimed in claim 1 in mixture with solvents or pharmaceutically acceptable excipients, in a form suitable to the intravenous, intramuscular, subcutaneous, intradermic administration or in ointment, gel or spray shape, suitable to the prophylaxis and the therapy of the plasmatic homeostasis.
